(19) United States
(12) Patent Application Publication
(10) Pub. No.: US 2022/0031968 A1
(43) Pub. Date: Feb. 3, 2022

(54) GAS INJECTION STABILIZATION DEVICE

(71) Applicant: NATIONAL CANCER CENTER, Goyang-si (KR)

(72) Inventors: Dae Kyung SOHN, Seoul (KR); Seong Chan PARK, Goyang-si (KR); Woo Sik EOM, Goyang-si (KR); Jae Hwan OH, Seoul (KR)

(73) Assignee: NATIONAL CANCER CENTER, Goyang-si (KR)

(21) Appl. No.: 17/277,543

(22) PCT Filed: Sep. 16, 2019
(86) PCT No.: PCT/KR2019/011914

§ 371 Date: Mar. 18, 2021

(30) Foreign Application Priority Data
Sep. 21, 2018 (KR) 10-2018-0114168

(51) Int. Cl. A61M 13/00

(52) U.S. Cl. CPC A61M 13/003; A61M 2202/0225

(57) ABSTRACT

The present disclosure relates to a gas injection stabilization device and comprises: a fixed volume part having a first gas space with a fixed volume; at least one variable volume part which is in communication with the fixed volume part, and which has a volume that varies due to a gas introduced to and discharged from the fixed volume part, and which has a second gas space connected to the first gas space; a gas supply part which is connected to the fixed volume part and receives a supply of a gas from an external gas supply device; and a gas discharge part which is connected to the fixed volume part and discharges a gas to an external surgical space.

### TECHNICAL FIELD

The present disclosure relates to a gas injection stabilization device that stabilizes a pressure change of an injected gas to secure space during surgery.

### BACKGROUND ART

Minimally invasive surgery is a method of making an incision with a size of 0.5 to 1.5 cm in 3 to 4 places to form a hole, and then inserting an endoscope equipped with a special camera and surgical instruments; it is important to secure a field of view in the minimally invasive surgery.

To this end, a gas such as carbon dioxide is injected into a body, and in the related art, when surgery is performed in a narrow space within the body, it is difficult to maintain a constant pressure, and thus, it is difficult to perform a stable surgery.

Therefore, during surgery, a device for maintaining stable pressure in inside of the body is required.

### DISCLOSURE

### Technical Problem

An object of the present disclosure is to provide a gas injection stabilization device for stabilizing a pressure change of a gas in a surgical space.

### Technical Solution

According to an aspect of the present disclosure, there is provide a gas injection stabilization device for reducing a pressure change of a gas injected into a body to secure a space during surgery, including: a fixed volume part having a first gas space with a fixed volume; at least one variable volume part which is in communication with the fixed volume part, and which has a volume that varies due to a gas introduced to and discharged from the fixed volume part, and which has a second gas space connected to the first gas space; a gas supply part which is connected to the fixed volume part and receives a supply of a gas from an external gas supply device; and a gas discharge part which is connected to the fixed volume part and discharges a gas to an external surgical space.

The gas supply part may comprise a supply channel through which a gas is introduced and moved and a supply end through which the introduced gas is discharged, and the gas discharge part may comprise a discharge end into which the gas in the first gas space is introduced and a discharge channel through which the gas moves and is discharged to the outside of the fixed volume part.

The supply channel may comprise a first supply channel connected to the outside, and a second supply channel branched from the first supply channel and having the supply end.

The second supply channel may comprise a first sub-channel having the supply end, and a second sub-channel.

The first supply channel, the first sub-channel, and the second sub-channel may have any one of a V shape, a Y shape, and a T shape.

The variable volume part may be provided as a pair of variable volume parts so that the fixed volume part is disposed therebetween, and the supply end of the first sub-channel may face one of the variable volume parts, and the supply end of the second sub-channel may discharge a gas toward the other of the variable volume parts.

The fixed volume part may be formed in a cylindrical shape.

### Advantageous Effects

According to the present disclosure, a gas injection stabilization device is provided to stabilize a pressure change of a gas injected to secure a stable space and a surgical field of view during surgery.

### DESCRIPTION OF DRAWINGS

FIG. 1 illustrates a gas injection stabilization device according to a first embodiment of the present disclosure.

FIG. 2 is a cross-sectional view of a fixed volume part along line 11-11′ of FIG. 1.

FIGS. 3a and 3b are views for describing a use of a gas injection stabilization device according to the first embodiment of the present disclosure.

FIGS. 4a to 4d illustrate supply channels according to second to fifth embodiments of the present disclosure.

FIGS. 5a to 5e are schematic diagrams of gas supply parts and dispositions of the gas discharge parts according to eighth to twelfth embodiments of the present disclosure.

FIGS. 6a and 6b each illustrate gas injection stabilization devices used in Experiment 1.

FIGS. 7a to 7c each illustrate pressure measurement results according to measurement points in Experiment 1.

FIGS. 8a to 8c each illustrate pressure measurement results at measurement points in Experiment 2.

FIGS. 9a to 9c each illustrate pressure measurement results according to measurement points in Experiment 3.

FIGS. 10a and 10b each illustrate gas injection stabilization devices used in Experiment 4.

FIGS. 11a to 11c each illustrate pressure measurement results according to measurement points in Experiment 4.

FIGS. 12a to 12c each illustrate gas injection stabilization devices used in Experiment 5.

FIGS. 13a to 13c each illustrate pressure measurement results according to measurement points in Experiment 5.

### MODE FOR DISCLOSURE

The above-described embodiments are examples for explaining the present disclosure, and the present disclosure is not limited thereto. A person with ordinary knowledge in the technical field to which the present disclosure belongs will be able to perform the present disclosure in various ways from the above-described embodiments, and thus, a scope of technical protection of the present disclosure should be regarded as belonging to the scope of the present disclosure with all variations and modifications.

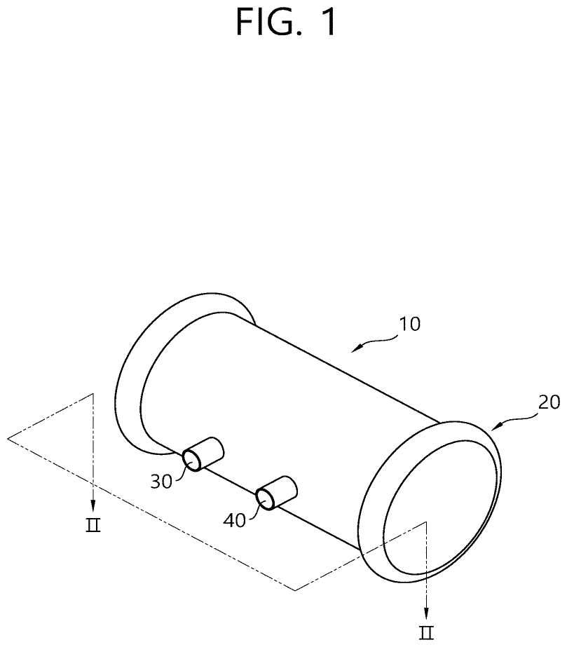

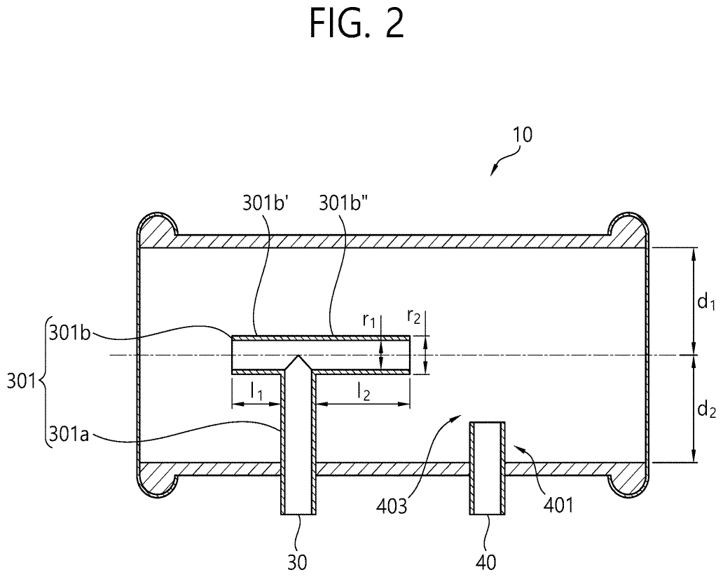

Hereinafter, a gas injection stabilization device 1 according to a first embodiment of the present disclosure will be described in more detail with reference to FIGS. 1 and 2.

FIG. 1 illustrates the gas injection stabilization device 1 according to the first embodiment of the present disclosure, and FIG. 2 is a cross-sectional view of a fixed volume part 10 along line II-II′ of FIG. 1.

As illustrated, the gas injection stabilization device 1 according to the first embodiment of the present disclosure comprises the fixed volume part 10 having a first gas space with a fixed volume, and a variable volume part 20 which is in communication with the fixed volume part 10, has a volume that varies due to a gas introduced to and discharged from the fixed volume part 10, and has a second gas space connected to the first gas space.

The fixed volume part 10 is a cylindrical form made of plastic material. However, the fixed volume part 10 is not limited thereto, and may be formed of various materials and shapes as long as it is connected to supply a gas to the variable volume part 20 well and has a fixed volume even when the gas is introduced thereinto or discharged therefrom.

The variable volume part 20 is provided as a pair of variable volume parts 20 so that the fixed volume part 10 is interposed therebetween. The variable volume part 20 is formed of a rubber material. However, the variable volume part 20 may be formed of other materials according an amount of the gas introduced thereinto or discharged therefrom as long as it is connected to the fixed volume part 10.

The fixed volume part 10 comprises a gas supply part 30 which receives a supply of a gas from an external gas supply device, and a gas discharge part 40 which is connected to the fixed volume part 10 and discharges a gas to an external surgical space. The gas supply part 30 and the gas discharge part 40 are disposed on a straight line parallel to an extension direction of the fixed volume part 10.

The gas supply part 30 comprises a supply channel 301 through which gas is introduced and moved, and a supply end 303 through which the introduced gas is discharged into the first gas space.

The supply channel 301 comprises a first supply channel 301a connected to the outside, and a second supply channel 301b branching from the first supply channel 301a and having the supply end 303.

The second supply channel 301b comprises a first sub-channel 301b′ and a second sub-channel 301b″ each having the supply end 303.

The gas discharge part 40 comprises a discharge end 403 through which the gas in the gas space is introduced and a discharge channel 401 through which the gas is moved and discharged to the outside of the fixed volume part 10.

The first sub-channel 301b′ and the second sub-channel 301b″ are each disposed to inject a gas toward different variable volume parts 20.

The first supply channel 301a, the first sub-channel 301b′, and the second sub-channel 301b″ have a T-shape. The gas discharged through the supply end 303 of the sub-channels 301b′ and 301b″ is located toward a center of a surface where the fixed volume part 10 and the variable volume part 20 communicate with each other. That is, lengths d1 and d2 of FIG. 2 are the same. However, the present disclosure is not limited thereto, and various embodiments are possible depending on an amount of gas and a type of the variable volume part 20.

In a case where the gas discharged through the supply end 303 is directed to a center of a surface where the fixed volume part 10 and the variable volume part 20 communicate with each other, even when the amount of gas is the same, the air can be effectively transferred from the fixed volume part 10 to the variable volume part 20, and an effect of reducing a pressure at a surgical site may be increased.

A length 11 of the first sub-channel 301b′ is longer than a length 12 of the second sub-channel 301b″, and the supply end 303 of the first sub-channel 301b′ is located more adjacent to the discharge end 403 than the second sub-channel 301b″. The present disclosure is not limited thereto, and the length 11 of the first sub-channel 301b′ and the length 12 of the second sub-channel 301b″ may be the same (not illustrated) or similar (not illustrated). When the length 12 of the second sub-channel 301b″ becomes longer, there is an effect of increasing a pressure distribution of the gas transferred to the variable volume part 20 located in an elongation direction.

The first gas space is elongated, and the second supply channel 301b is disposed parallel to the extension direction of the first gas space. A cross-sectional area of a gas channel of the second supply channel 301b is constant (r1=r2 in FIG. 2). However, the present disclosure is not limited thereto, and various embodiments are possible.

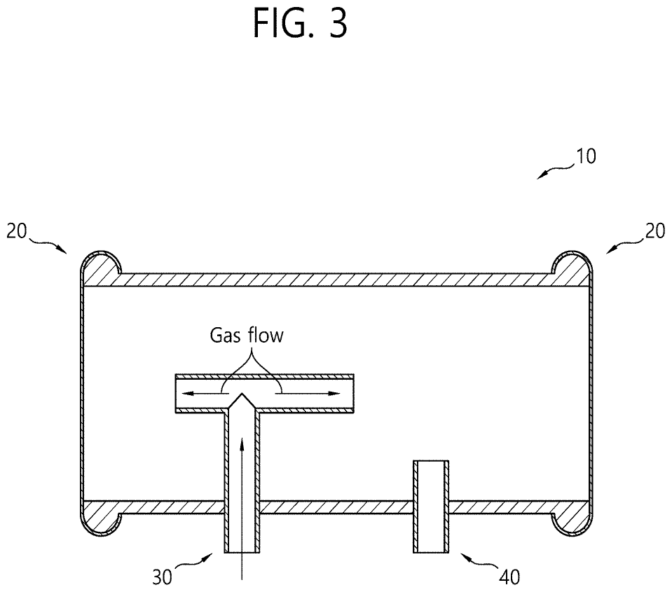

A method of using the above-described gas injection stabilization device 1 will be described with reference to FIGS. 3a and 3b.

FIGS. 3a and 3b are views for describing a use of the gas injection stabilization device 1 according to the first embodiment of the present disclosure.

A user (medical staff) connects the gas supply part 30 to an external gas supply device, and connects the gas discharge part 40 to a trocar for introducing gas to a surgical site. The user proceeds with the surgery while supplying gas to the surgical space (refer to FIG. 3a).

In this process, the gas supplied to the gas supply part 30 moves to the variable volume part 20, and the variable volume part 20 swells to a certain degree to form the second gas space.

In this case, gas may be irregularly supplied to the gas injection stabilization device 1 at a high pressure (excessive amount) depending on the operation of the external supply device, and the variable volume part 20 swells further as illustrated in FIG. 3b, and thus, a volume of the second gas space increases. In this way, the variable volume part 20 stabilizes a pressure change of the gas while repeating the expansion and contraction, and the gas whose pressure change is stabilized is discharged through the gas discharge part 40 to secure a space at a surgical site, and thus, the pressure change in the surgical space is prevented.

In the gas injection stabilization device 1 according to the present disclosure, a basic volume is secured since the fixed volume part 10 functioning as a buffer is provided, and it is possible to reduce the pressure change in the surgical space since the variable volume part 20 that stabilizes the pressure change of the gas while expanding and contracting is provided.

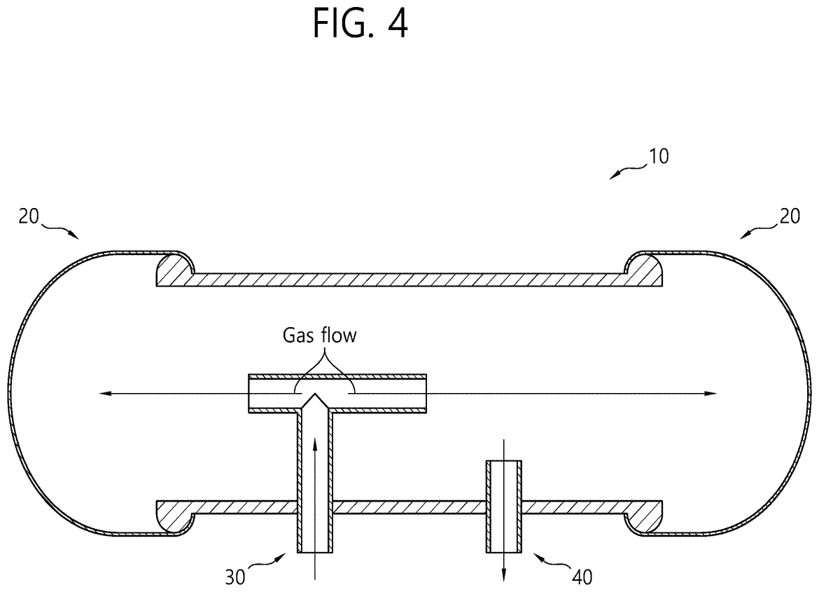

FIGS. 4a to 4d illustrate shapes of supply channels 301 according to second to fifth embodiments of the present disclosure.

As illustrated in FIGS. 4a and 4b, the first supply channel 301a, the first sub-channel 301b′, and the second sub-channel 301b″ may have a Y shape and a V shape in each of the second and third embodiments. In addition, as illustrated in FIGS. 3c and 3d, in the fourth embodiment, only the first sub-channel 301b′ may be formed, and in the fifth embodiment, the second supply channel 301b may be omitted.

Meanwhile, in a cross-sectional area of the second supply channel 301b, the sixth embodiment (not illustrated) may be provided, in which a diameter of the second supply channel 301b becomes narrower toward the supply end 303, that is, r1 is greater than r2. Moreover, the seventh embodiment (not illustrated) may be provided, in which the diameter of the second supply channel 301b becomes wider toward the supply end 303, that is, r1 is smaller than r2.

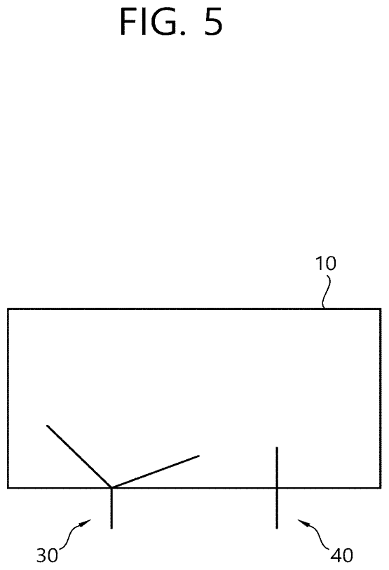

FIGS. 5a to 5e illustrate dispositions of the gas supply part 30 and the gas discharge part 40 according to eighth to twelfth embodiments of the present disclosure.

As illustrated in 5a to 5e, unlike the fixed volume part 10 of the first embodiment of the present disclosure in which the gas supply part 30 and the gas discharge part 40 are disposed side by side, various embodiments may be provided in which the gas supply part 30 and the gas discharge part 40 face each other.

A surgical method using the gas injection stabilization device according to the present disclosure is as follows.

The user (medical staff) connects the gas supply part 30 to the external gas supply device, and connects the gas discharge part 40 to the trocar for introducing gas to the surgical site. The user proceeds with the surgery while supplying a gas to the surgical space (refer to FIG. 3a). In this process, the gas supply part 30 reduces the pressure change at the surgical site, and thus, the surgery can be easily performed.

Hereinafter, the present disclosure will be described in more detail through experimental examples. These experimental examples are only for describing the present disclosure in more detail, and the scope of the present disclosure is not limited by these experimental examples according to a gist of the present disclosure.

### Experiment 1 (Experimental Examples 1 to 2): Measurement of Pressure Distribution According to Presence or Absence of Second Supply Channel 301b

1) Experiment Method

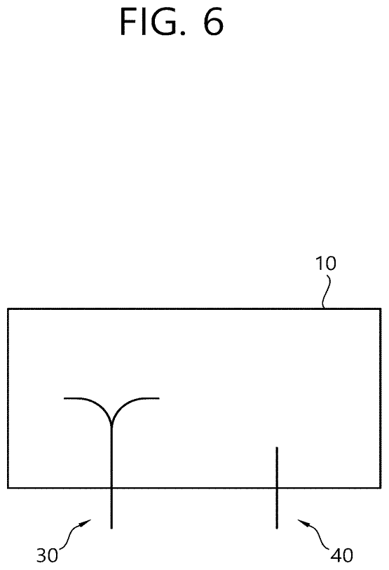

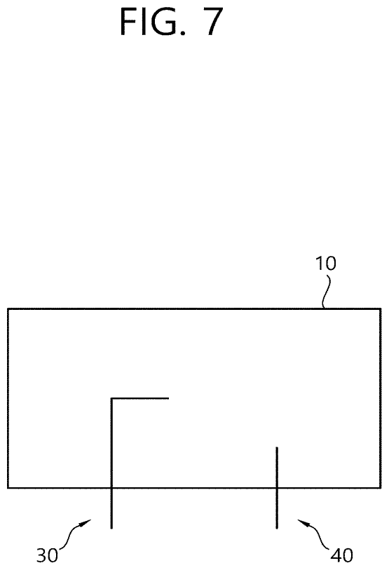

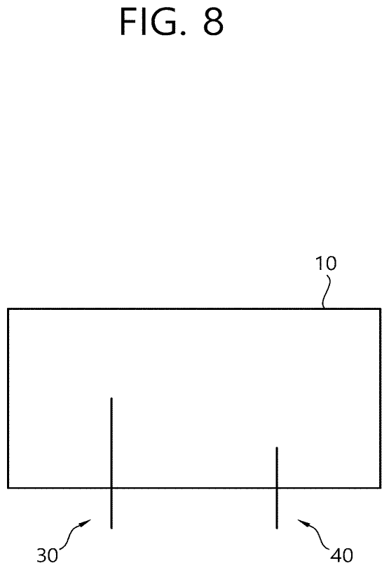

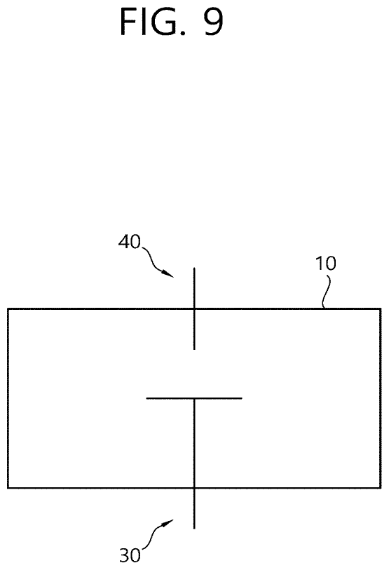

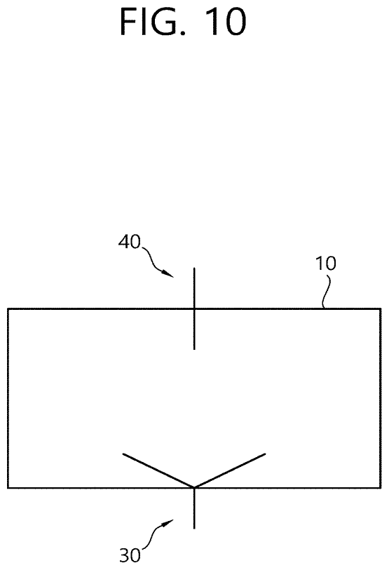

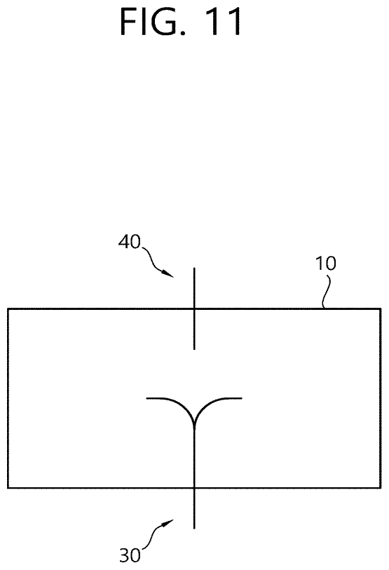

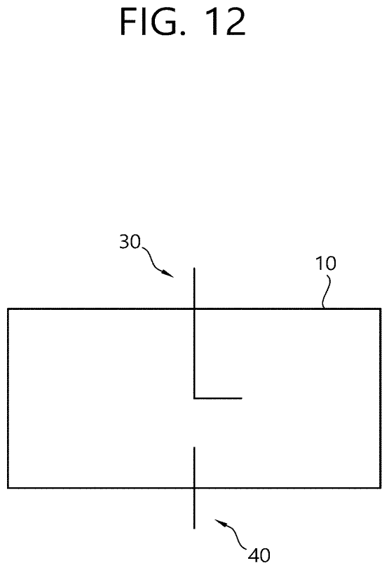

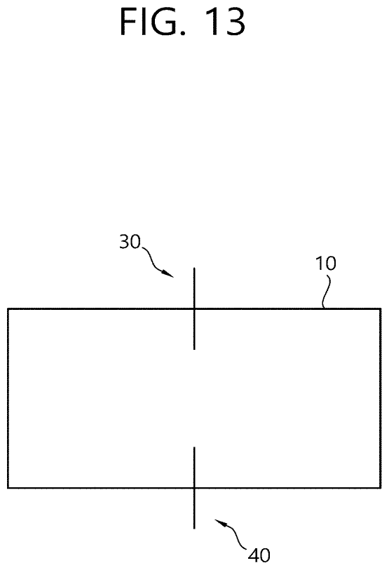

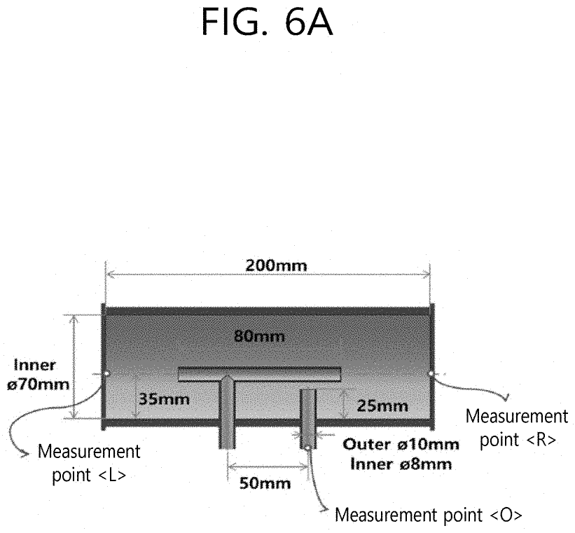

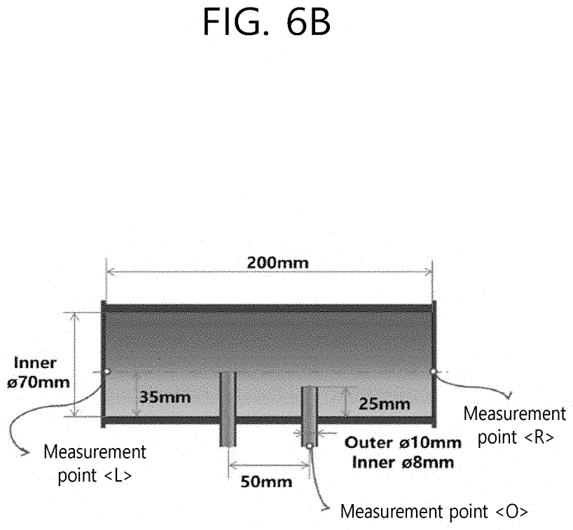

In Experimental Example 1, the gas injection stabilization device 1 according to the first embodiment of the present disclosure was used, in which the second supply channel 301b was formed in the cylindrical fixed volume part 10 made of a plastic material having a diameter of 70 mm and a height of 200 mm and a T-shaped gas supply part 30 was provided, and specific values are as illustrated in FIG. 6a.

In Experimental Example 2, the gas injection stabilization device 1 according to the fifth embodiment of the present disclosure was used, in which the second supply channel 301b was not formed, and specific values are as illustrated in FIG. 6b.

In each experimental example, a 40L High Flow Insufflator connected to a container containing CO₂ gas was connected to the first supply channel 301a of the gas injection stabilization device 1, a patient's body pack instead of a patient's abdominal cavity was connected to the discharge channel 401, and a device capable of monitoring pressure levels and changes to the patient's body pack was connected.

The pressure distribution according to gas injection from an external device was measured by flow analysis through computational fluid dynamics (cFd).

In addition, in L (left) and R (Right) sides of centers at which the fixed volume part 10 and both variable volume parts 20 toward which the gas discharged from the supply end 303 was directed met each other and a center point (output) of the discharge channel through which the gas was discharged to the outside, the pressure was measured when the gas was introduced into L (left), R (Right), and center point.

2) Result

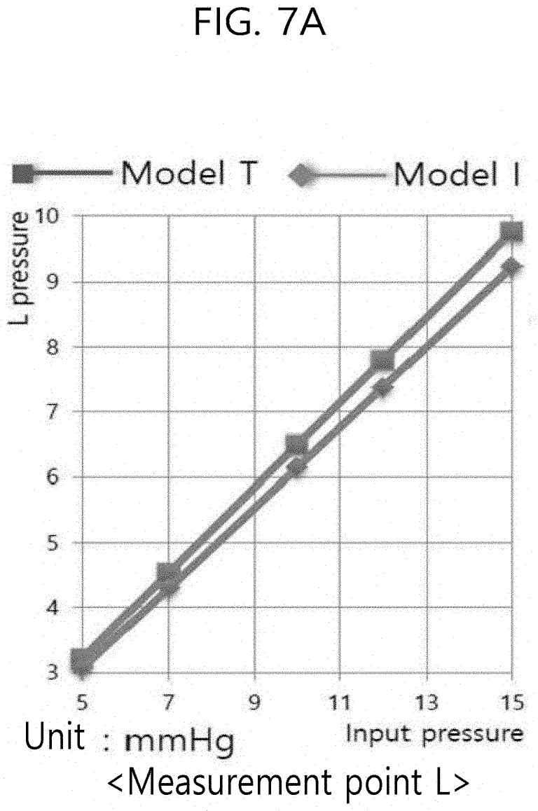

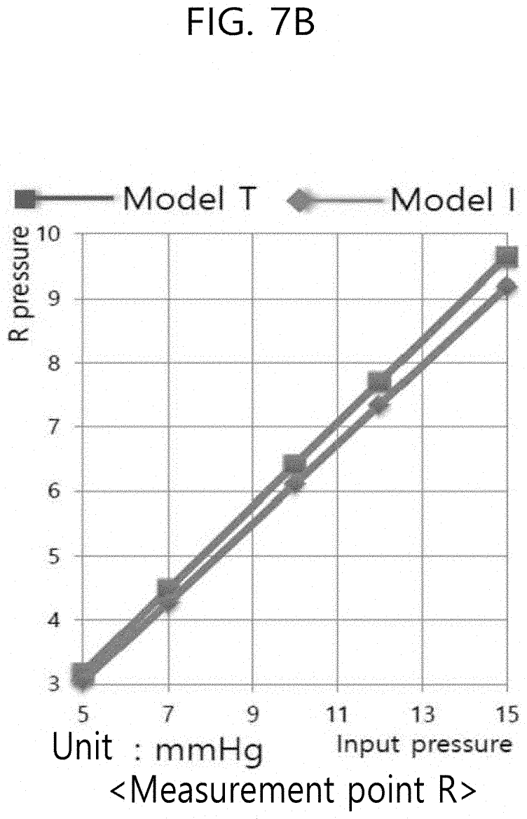

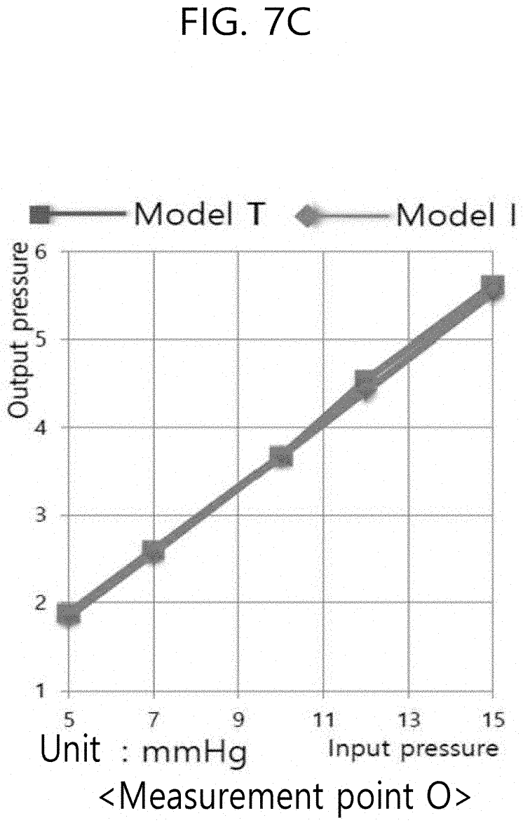

FIGS. 7a to 7c illustrate pressure measurement results according to the measurement points L, R, and O in the first embodiment (MODEL T) and the fifth embodiment (MODEL I) in Experiment 1, respectively.

As illustrated in FIGS. 7a to 7c, compared to the fifth embodiment, in the first embodiment, the pressure distribution from the fixed volume part 10 to the variable volume part 30 is made more efficiently, and it can be seen that the pressure of L and R is high. That is, in the fifth embodiment, there is no difference in the gas pressure of the output (measurement point O) discharged to the outside, and thus, the gas pressure can be efficiently adjusted. However, in the first embodiment having the T-shaped gas supply part in which the second supply channel 310b is formed, it can be seen that the gas in the variable volume part 30 is efficiently transferred.

### Experiment 2 (Experimental Examples 3 to 5): Measurement of Pressure Distribution Along Length of Second Sub-Channel 301b″

1) Experiment Method

In Experimental Example 3, a gas injection stabilization device 1 was used in which the cylindrical fixed volume part 10 made of a plastic material having a diameter of 70 mm and a height of 200 mm and the sub-channel 301b″ having a length of 30 mm from the center of the first sub-channel 301b′ were provided, and in Experimental Example 4, a gas injection stabilization device 1 was used in which the cylindrical fixed volume part 10 and the sub-channel 301b″ having a length of 60 mm from the center were provided. In Experimental Example 5, a gas injection stabilization device 1 was used in which the cylindrical fixed volume part 10 and the sub-channel 301b″ having a length of 95 mm from the center were provided. The pressure distribution of the fixed volume part 10 and the pressure levels of the measurement points L, R, and O were measured in the same manner as in Experiment 1.

2) Result

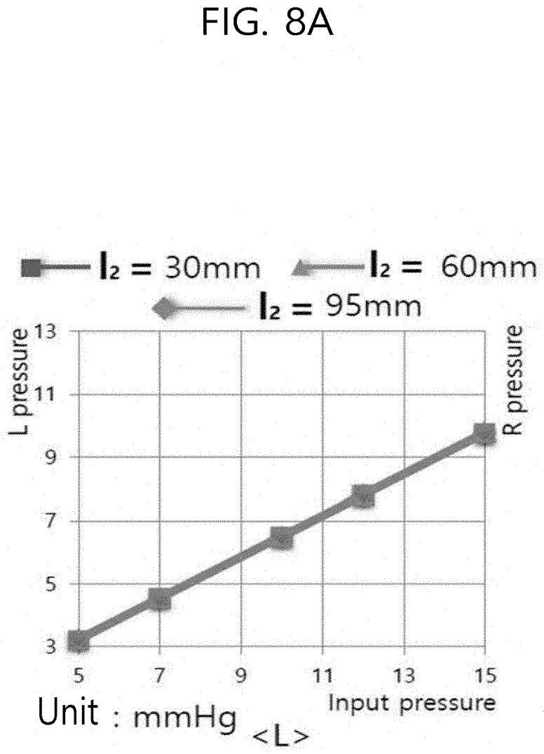

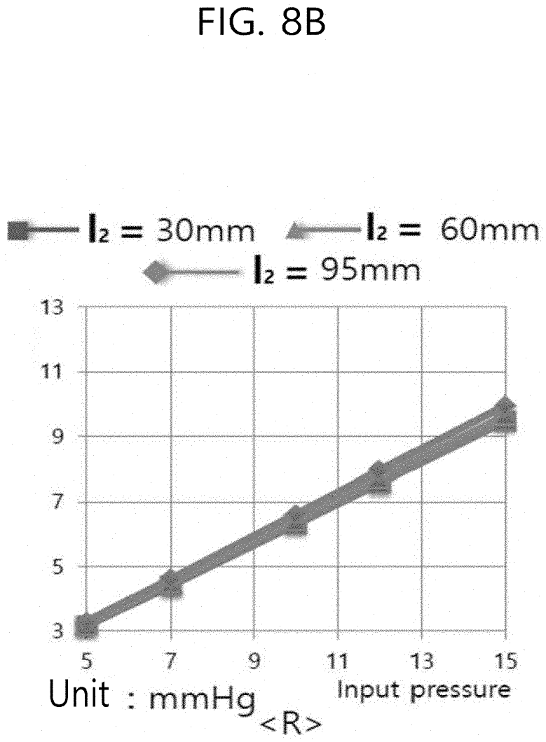

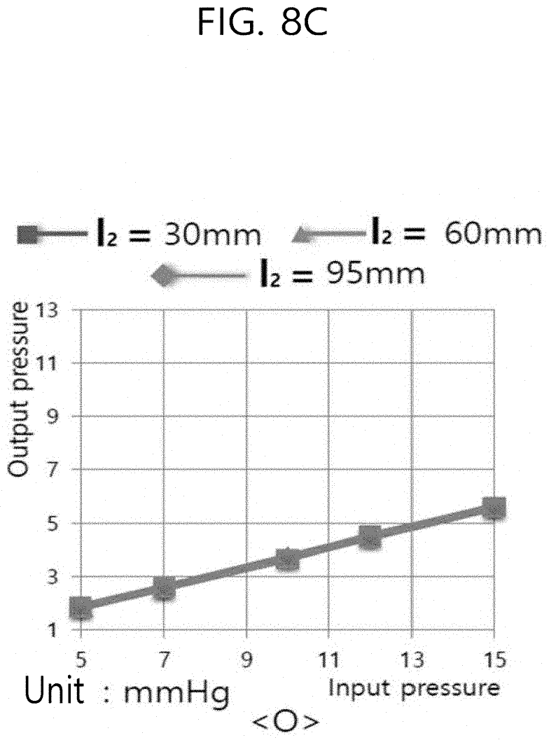

FIGS. 8a to 8c are graphs illustrating the pressure measurement results at the measurement points L, R, and O in Experiment 2.

As illustrated, it can be seen that when the length of the second sub-channel 301b″ at the measurement point R is 60 mm rather than 30 mm, the pressure transfer is more effective, and when the length is 95 mm rather than 60 mm, the pressure transfer is more effective. All pressure distributions at the other points are similar. That is, as the length of the second supply channel 301b becomes longer, the distance to the variable volume part 20 becomes closer, and thus, the gas movement is smooth. Therefore, the efficiency of pressure transfer can be improved as it approaches the variable volume part 20.

### Experiment 3 (Experimental Examples 6 to 8): Measurement of Pressure Distribution According to Shape of Supply End

1) Experiment Method

In Experimental Example 6, a gas injection stabilization device 1 of the first embodiment of the present disclosure in which the gas channel cross-sectional area of the second supply channel 301b was constant (r1=r2 in FIG. 2) was used, and in Experimental Example 7, a gas injection stabilization device 1 of the sixth embodiment (r1>r2 in FIG. 2, not illustrated) in which the diameter of the second supply channel 301b was narrower toward the supply end 303 was used. In Experimental Example 8, a gas injection stabilization device 1 of the seventh embodiment (r1<r2 in FIG. 2, not illustrated) in which the diameter of the second supply channel 301b was wider toward the supply end 303 was used. The pressure distribution of the fixed volume part 10 and the pressure levels of the measurement points L, R, and O were measured in the same manner as in Experiment 1.

2) Result

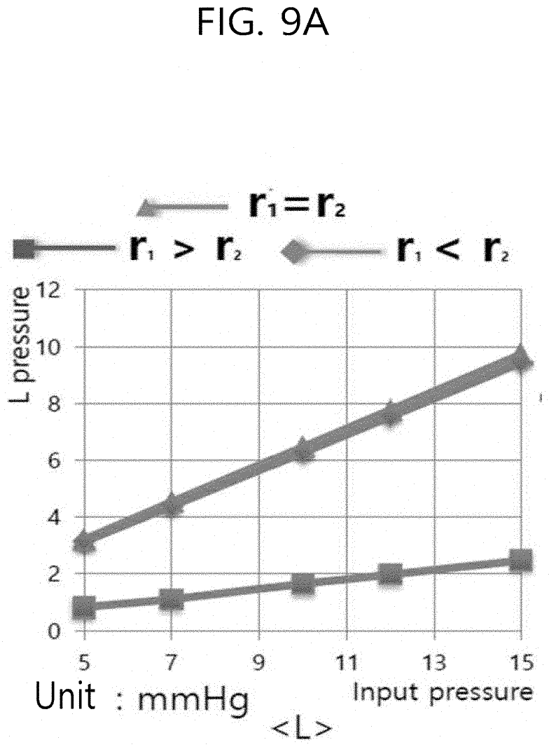

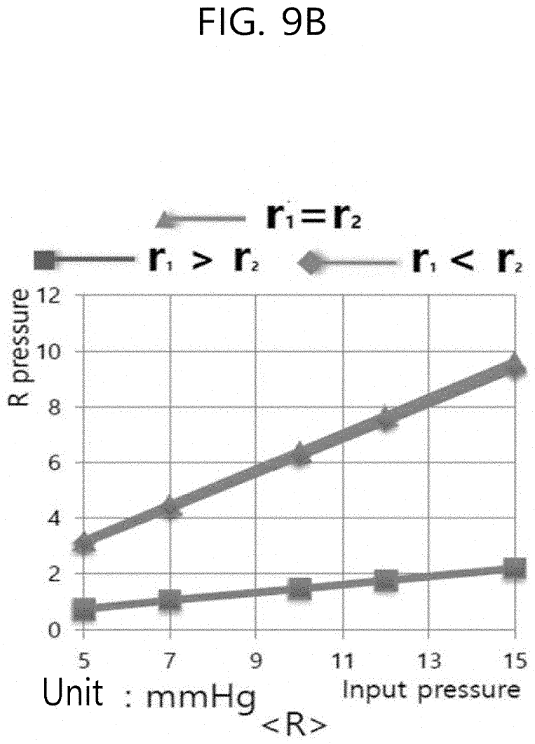

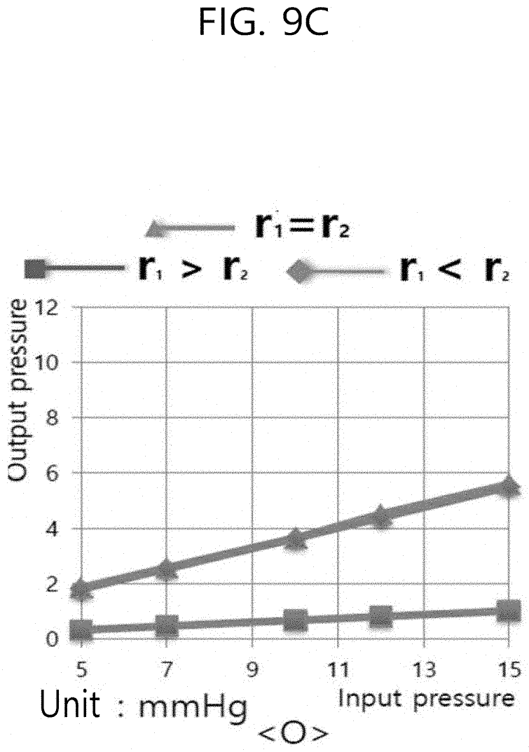

FIGS. 9a to 9c illustrate measurement results of Experiment 3 for the pressure measurements according to the measurement points L, R, and O.

As illustrated, it is confirmed that the pressure transfer is more efficient in the sixth and seventh embodiments according to the present disclosure than in the first embodiment. It is determined that this is because in the case of the sixth embodiment, the supply end 303 is narrowed to reduce the amount of gas discharged, and in the case of the seventh embodiment, the supply end 303 is widened such that the pressure of the discharged gas is lower than that of the supplied gas.

### Experiment 4 (Experimental Examples 9 to 10): Measurement of Pressure Distribution According to Position of Gas Discharge Part 40

1) Experiment Method

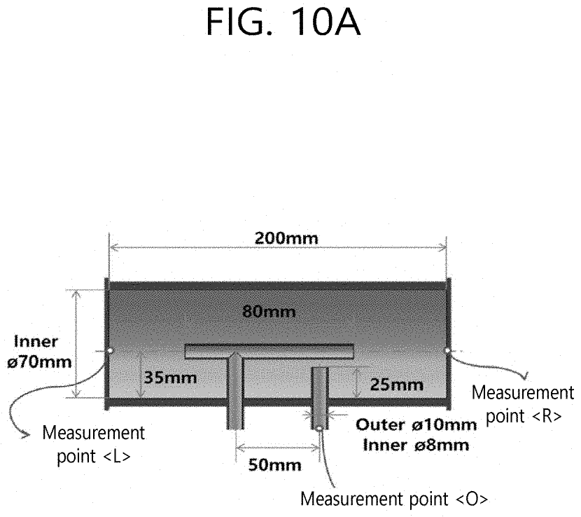

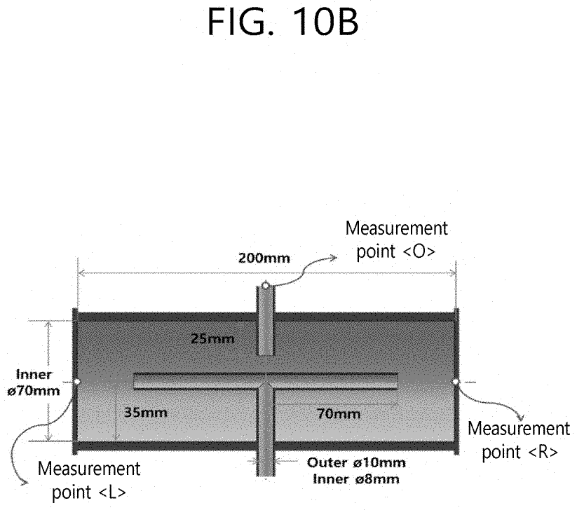

In Experimental Example 9, the gas injection stabilization device 1 according to the first embodiment of the present disclosure was used in which the gas supply part 30 and the gas discharge part 40 were disposed side by side in the fixed volume part 10, and the specific values are as illustrated in FIG. 10a.

In Experimental Example 10, the gas injection stabilization device 1 according to the eighth embodiment of the present disclosure was used in which the gas supply part 30 and the gas discharge part 40 were disposed to face each other, and the specific values are as illustrated in FIG. 10b.

The pressure distribution of the fixed volume part 10 and the pressure levels of the measurement points L, R, and O were measured in the same manner as in Experiment 1.

2) Result

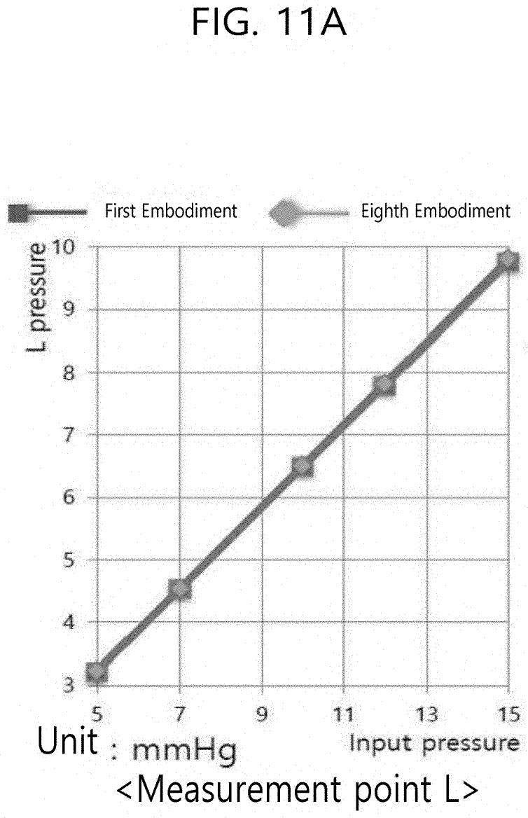

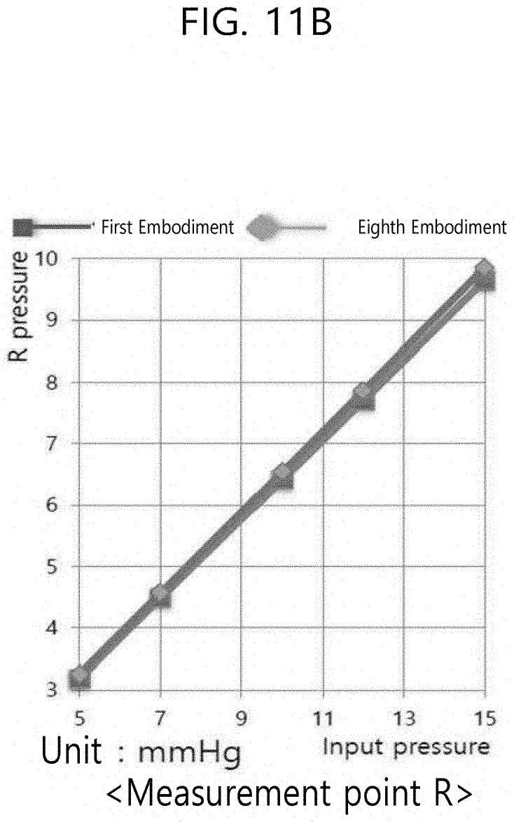

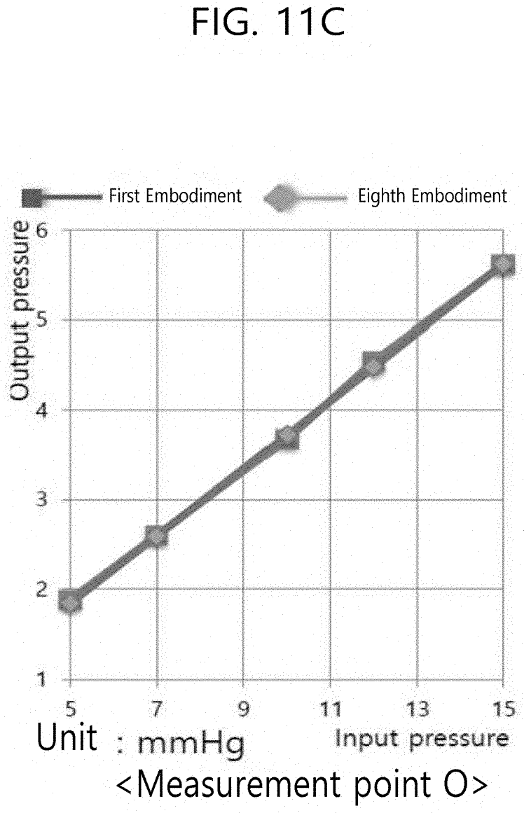

FIGS. 11a to 11c illustrate the pressure measurement results according to the measurement points L, R, and O in Experiment 4.

As illustrated in FIGS. 11a to 11c, in the first embodiment and the eighth embodiment, it can be seen that there is no difference in the pressure distributions of the fixed volume part 10 and the pressure levels at the measurement point L, R, and O. Therefore, it can be seen that the position of the gas discharge part 40 does not have a significant effect.

### Experiment 5 (Experimental Examples 11 to 13): Measurement of Pressure Distribution According to Shape of Second Supply Channel 301b

1) Experiment Method

In Experimental Example 11, the gas injection stabilization device 1 according to the first embodiment of the present disclosure was used, in which the second supply channel 301b was formed in the cylindrical fixed volume part 10 made of a plastic material having a diameter of 70 mm and a height of 200 mm and a T-shaped gas supply part 30 was provided, and specific values are as illustrated in FIG. 6a.

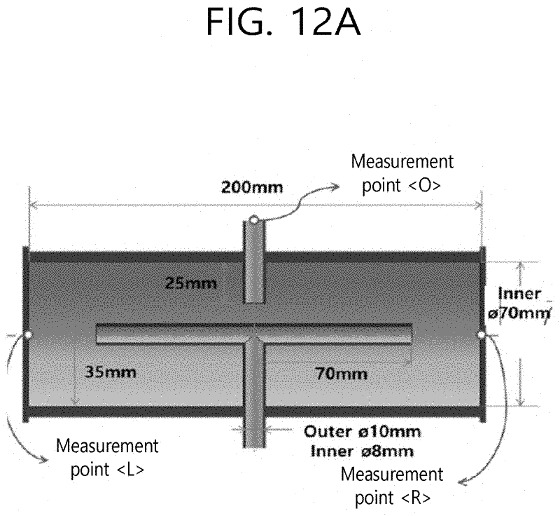

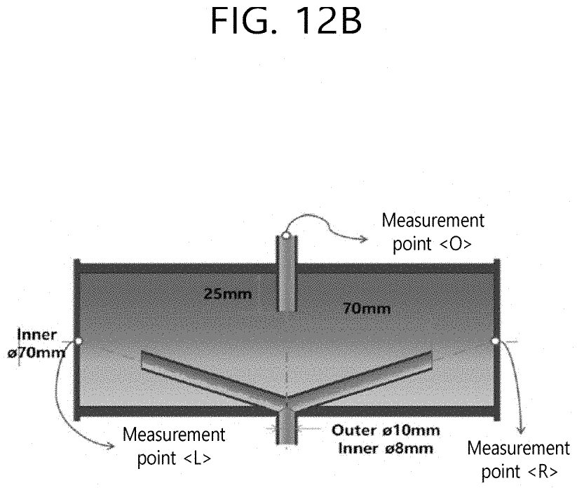

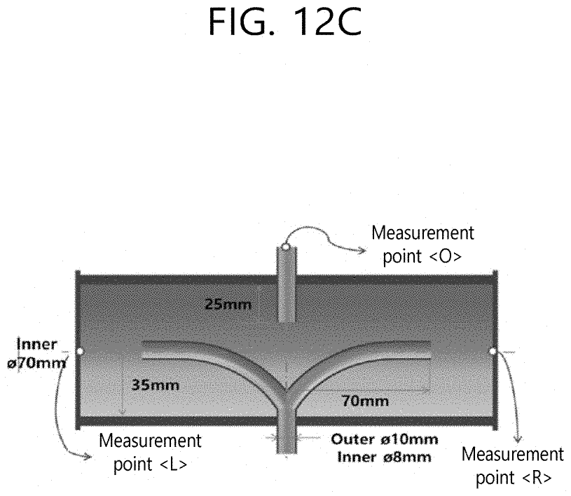

In Experimental Example 12, the gas injection stabilization device 1 according to the ninth embodiment of the present disclosure including a V-shaped gas supply part 30 was used, and the specific values are as illustrated in FIG. 12b.

In Experimental Example 13, the gas injection stabilization device 1 according to the tenth embodiment of the present disclosure including a U-shaped gas supply part 30 was used, and the specific values are as illustrated in FIG. 12c.

The device was designed so that the gas introduced out through the supply end 303 faced toward the center of the surface where the fixed volume part 10 and the variable volume part 20 communicated with each other, and the pressure distribution of the fixed volume part 10 and the pressure levels of the measurement points L, R, and O were measured in the same manner as in Experiment 1.

2) Result

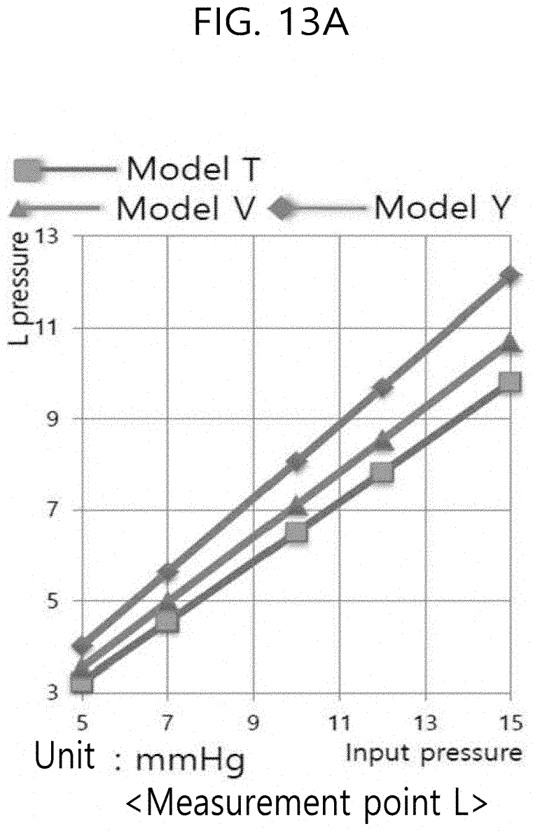

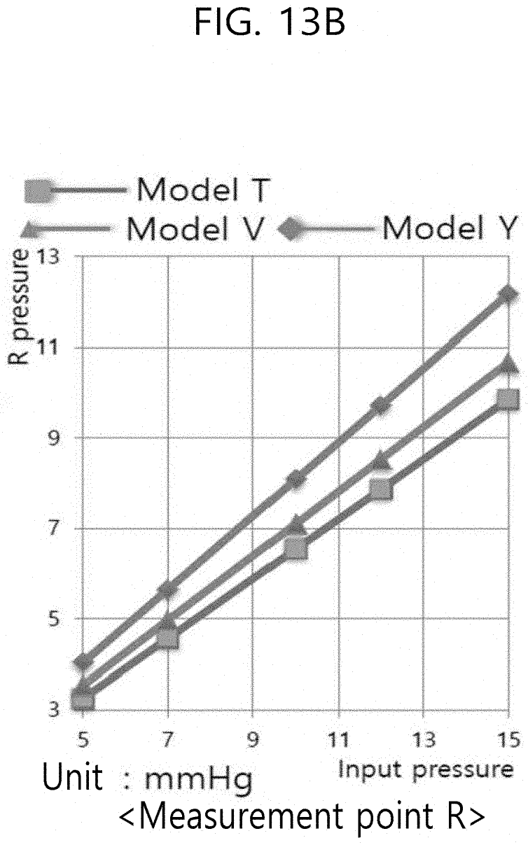

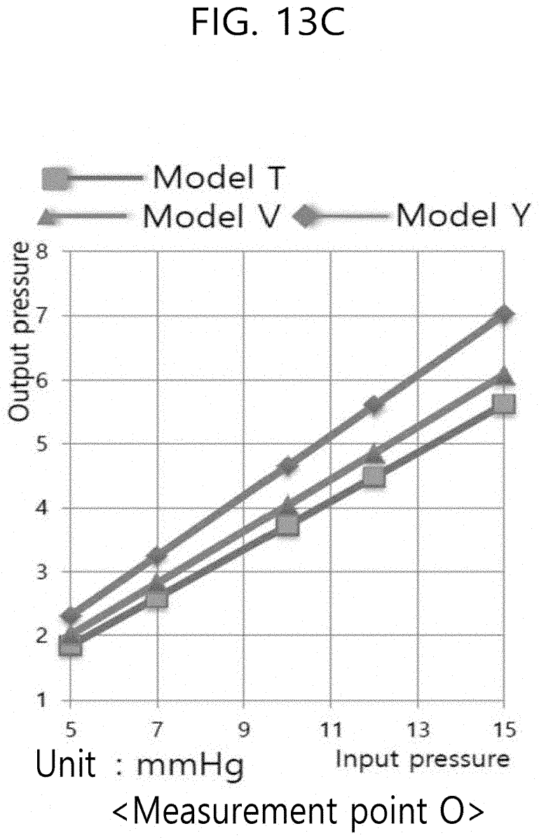

FIGS. 13a to 13c illustrate the pressure measurement results according to the measurement points in Experiment 5.

As illustrated in FIGS. 13a to 13c, it can be seen that the pressure transfer of the ninth embodiment is more effective than that of the eighth embodiment and the pressure transfer of the tenth embodiment is more effective than that of the ninth embodiment. It is determined that this is because the efficiency of pressure transfer can be improved by changing a direction of the kinetic energy of a gas as slowly as possible.

In the gas injection stabilization device 1 according to the present disclosure, a basic volume is secured since the fixed volume part 10 functioning as a buffer is provided, and it is possible to reduce the pressure at the surgical site even when the amount of gas changes since the variable volume part 20 whose volume changes according to the amount of gas is provided. Moreover, since the second supply channel which branches from the first supply channel 301a connected to the outside and has the first sub-channel 301b′ having the first sub-channel 301b′ and the second sub-channel 301b″, the second sub-channel 310b″ having the length longer than that of the first sub-channel 301b′, and the second supply channel 301b having the gas channel whose cross-sectional area is constant are provided, the effects are improved, and the gas can stably flow and a continuous pressure can be maintained inside the body during surgery.

1. A gas injection stabilization device for reducing a pressure change of a gas injected to a body to securing a space during surgery, comprising:
a fixed volume part having a first gas space with a fixed volume;
at least one variable volume part which is in communication with the fixed volume part, and which has a volume that varies due to a gas introduced to and discharged from the fixed volume part, and which has a second gas space connected to the first gas space;
a gas supply part which is connected to the fixed volume part and receives a supply of a gas from an external gas supply device; and
a gas discharge part which is connected to the fixed volume part and discharges a gas to an external surgical space.

2. The gas injection stabilization device of claim 1, wherein the gas supply part comprises a supply channel through which a gas is introduced and moved and a supply end through which the introduced gas is discharged, and
the gas discharge part comprises a discharge end into which the gas in the first gas space is introduced and a discharge channel through which the gas moves and is discharged to the outside of the fixed volume part.

3. The gas injection stabilization device of claim 1, wherein the supply channel comprises
a first supply channel connected to the outside, and
a second supply channel branched from the first supply channel and having the supply end.

4. The gas injection stabilization device of claim 3, wherein the second supply channel comprises
a first sub-channel having the supply end, and
a second sub-channel.

5. The gas injection stabilization device of claim 4, wherein the first supply channel, the first sub-channel, and the second sub-channel have any one of a V shape, a Y shape, and a T shape.

6. The gas injection stabilization device of claim 4, wherein the variable volume part is provided as a pair of variable volume parts so that the fixed volume part is disposed therebetween, and
the supply end of the first sub-channel faces one of the variable volume parts, and the supply end of the second sub-channel discharges a gas toward the other of the variable volume parts.

7. The gas injection stabilization device of claim 1, wherein the fixed volume part is formed in a cylindrical shape.
